(19) United States
(12) Patent Application Publication
(10) Pub. No.: US 2024/0081937 A1
(43) Pub. Date: Mar. 14, 2024

(54) ADJUSTABLE SEAL FOR FLUID MANAGEMENT SYSTEM

(71) Applicant: Washington University, St. Louis, MO (US)

(72) Inventors: Mohamed ZAYED, St. Louis, MO (US); Chase HARTQUIST, St. Louis, MO (US); Halle LOWE, St. Louis, MO (US); John CASHIN, St. Louis, MO (US)

(21) Appl. No.: 18/260,354

(22) PCT Filed: Jan. 10, 2022
(86) PCT No.: PCT/US2022/011761

§ 371 Date: Jul. 5, 2023

Related U.S. Application Data
(60) Provisional application No. 63/136,088, filed on Jan. 11, 2021.

(51) Int. Cl. A61B 90/00; A61M 16/04; A61M 16/06

(52) U.S. Cl. CPC A61B 90/05; A61M 16/0488; A61M 16/06

(57) ABSTRACT

An adjustable seal includes a first ring extending about an axis and including a plurality of first teeth. The adjustable seal also includes a second ring extending about the axis and including a plurality of second teeth. The first ring and the second ring cooperatively defining a passage extending along the axis. The adjustable seal has a locked configuration in which the second teeth engage the first teeth and prevent movement of the first ring relative to the second ring, and an unlocked configuration in which the first ring is able to rotate relative to the second ring. The adjustable seal also includes a flexible member attached to the first ring and the second ring. Rotation of the first ring relative to the second ring in the unlocked configuration of the adjustable seal causes the flexible member to extend across at least a portion of the passage defined by the first ring and the second ring.

### CROSS-REFERENCE TO RELATED APPLICATION

This application claims priority to U.S. Provisional Application No. 63/136,088 filed Jan. 11, 2021, the content of which is incorporated herein by reference in its entirety.

### FIELD OF THE DISCLOSURE

The present disclosure generally relates to adjustable seals or valves for air or fluid management systems such as airway management systems, endoscopic systems, laparoscopic systems, liquid tubing systems, and vascular sheaths.

### BACKGROUND OF THE DISCLOSURE

Airway management systems provide life-saving treatment for patients especially for patients with respiratory distress. For example, critically ill patients in the intensive care unit (ICU) often require positive pressure ventilation and oxygen delivery through an endotracheal (ET) tube, laryngeal mask airway (LMA), or bag mask valve (BMV). However, timely and appropriate management of patients with respiratory distress such as COVID-related acute respiratory distress syndrome (ARDS) may be hindered by reluctance of providers to initiate early intubation or alternatives such as LMA ventilation. Critically ill COVID patients with high oxygen requirements often deteriorate rapidly and require mechanical ventilation within 1-3 days. Current recommendations are to maintain patients on continuous positive airway pressure (CPAP) for as long as possible using the lowest effective pressures (e.g. 5-10 cm H₂O). For patients with higher oxygen requirements, noninvasive modalities are recommended rather than proceeding directly to intubation.

Medical providers may be reluctant to treat patients afflicted by COVID-19 or other highly transmittable respiratory illnesses in need of time-critical diagnostic or therapeutic bronchoscopy—an essential pulmonary procedure that is seldom performed unless highly indicated in a pandemic scenario. Mitigation of exposure to pathogens associated with the pandemic is crucial to healthcare infection prevention and control. However, existing ventilatory devices and airway management systems do not provide for preventing inadvertant release of respiratory viral droplets and aerosolized particles expelled from patient airways. For example, the LMA's may not be sealed around a ET tube during tube placement. When treating persons with known or suspected COVID-19, providers should don full contact and airborne personal protective equipment (PPE), including a fit-tested N95 respirator mask, a powered air purifying respirator (PAPR), and an isolation suit. However, the supply of PPE may be limited, in particular as COVID-19 infections surge in the course of the pandemic. Accordingly, simple to use and readily available seals for airway management systems can be life-saving and have a profound effect on public health during a pandemic.

Vascular sheaths may be used to encase or guide devices for medical procedures. Typically, the vascular sheaths include a valve at an interface that allows materials to be passed into the sheath and regulate fluid flow through the vascular sheaths. However, at least some vascular sheaths do not allow for manual adjustment relative to a material being passed into the sheath. As a result, the valve interface may leak and cause unnecessary bleeding. Some vascular sheaths includes adjustable valves that utilize pneumatic compress across the inlet and onto devices being advanced into the sheath. The pneumatic compression interface provides pressure and friction on the material being passed into the sheath and alter the ability of passing the device through the sheath for the medical procedure. In addition, the friction provided by pneumatic compression interfaces alter the haptic feedback which is necessary for proper use of a medical device during endovascular procedures. Accordingly, there is a need for valve or seal systems that provide a reduced friction force on the inserted devices in comparison to existing systems and allow manual adjustment of the valves to reduce risk of bleeding during endovascular procedures.

Endoscopic and laparoscopic procedures are performed by placing ports into compartments within the body of the patient. The ports are then insufflated with gas to expand the compartment and provide adequate space for visualization of critical structures. The insufflated compartments also facilitate the advancement of surgical devices to perform endoscopic and/or laparoscopic procedures. At least some laparascopic and endoscopic ports include valve systems that are not manually adjustable. Accordingly, laparascopic and endoscopic ports with pre-selected diameters much be chosen to facilitate passage of specific size instruments. However, large structures that are required to be removed from the insaflated body cavities may not fit through the valves in the ports. In addition, sometimes port access may lost during the medical procedure. As a result, the surgeon may have to make a surgical incision to facilitate the removal of resected structures. A manually adjustable laparascopic/endoscopic port valve could allow for insufflation of body cavities with gas, facilitate passage of different diameter tools while maintaining cavity insufflation, and facilitate the removal of resected structures without lose of port access or creation of a larger incision.

In addition, seals are also used outside the medical field in fluid management systems or other applications. Fluid systems typically include one or more valves or seals to regulate fluid flow. Valves or seals may have a port with a set diameter that is openable to allow a set fluid flow rate. In addition, at least some known seals or valves may be expensive to manufacture, prone to failure, and/or difficult to adjust. Accordingly, improved valves or seals would provide numerous advantages in many industries that utilize fluid management systems or other applications and not just in the medical field. For example, a manually adjustable valve could help modulate the passage of different volumes and rates of fluid flow through the fluid systems in a controlled fashion. The manually adjustable valve could also facilitate uni- or bi-directional flow.

### BRIEF DESCRIPTION OF THE DISCLOSURE

In one aspect, an adjustable seal includes a first ring extending about an axis and including a plurality of first teeth. The adjustable seal also includes a second ring extending about the axis and including a plurality of second teeth. The first ring and the second ring cooperatively define a passage extending along the axis. The adjustable seal has a locked configuration in which the second teeth engage the first teeth and prevent movement of the first ring relative to the second ring, and an unlocked configuration in which the first ring is able to rotate relative to the second ring. The adjustable seal also includes a flexible member attached to the first ring and the second ring. Rotation of the first ring relative to the second ring in the unlocked configuration of the adjustable seal causes the flexible member to extend across at least a portion of the passage defined by the first ring and the second ring.

In another aspect, an airway management system includes a mask sized to cover a nose and mouth of a person. The mask defines at least one passage sized to receive an instrument. The at least one passage extends along an axis. The airway management system also includes at least one seal attached to the mask for selectively sealing the at least one passage. The at least one seal includes a first ring extending around the axis, a second ring extending around the axis, and a flexible member attached to the first ring and the second ring. Rotation of the first ring relative to the second ring causes the flexible member to extend across at least a portion of the passage defined by the mask and seal against the instrument if the instrument is positioned in the passage.

In another aspect, a method of sealing a passage includes positioning an adjustable seal in a passage. The adjustable seal includes a plurality of concentric rings and a flexible member attached to the concentric rings. The method also includes rotating at least one of the concentric rings relative to another of the concentric rings to cause the flexible member to extend across at least a portion of the passage.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a perspective view of one suitable embodiment of an adjustable seal of the present disclosure in a first, opened configuration.

FIG. 2 is a perspective view of the adjustable seal of FIG. 1 in a second, partly opened configuration.

FIG. 3 is a perspective view of the adjustable seal of FIG. 1 in a third, partly closed configuration.

FIG. 4 is a perspective view of a portion of the adjustable seal of FIG. 1, illustrating a first ring of the adjustable seal engaged with a second ring of the adjustable seal to lock the adjustable seal.

FIG. 5 is a top view of the first and second rings of FIG. 4.

FIG. 6 is a perspective view of the first and second rings of FIG. 4, illustrating the first and second rings separated to unlock the adjustable seal.

FIG. 7 is a side view of the first and second rings of FIG. 6.

FIG. 8 is a top view of the first ring of FIG. 4, illustrating a plurality of engagement teeth of the first ring.

FIG. 9 is a top view of the second ring of FIG. 4, illustrating a plurality of engagement teeth of the second ring.

FIG. 10 is a perspective view of one suitable embodiment of an airway management system including a mask and a plurality of adjustable seals for selectively closing passages through the mask.

FIG. 11 is a top view of a portion of the airway management system of FIG. 10, illustrating the adjustable seals in an opened configuration.

FIG. 12 is a top view of a portion of the airway management system of FIG. 10, illustrating the adjustable seals in a partly open configuration.

FIG. 13 is a top view of a portion of the airway management system of FIG. 10, illustrating the adjustable seals in a closed configuration.

FIG. 14 is a top view of a portion of the airway management system of FIG. 10, illustrating one of the adjustable seals in an open configuration and another of the adjustable seals in a closed configuration.

FIG. 15 is a perspective view of a portion of the airway management system of FIG. 10, illustrating an interface for the adjustable seals.

FIG. 16 is a perspective view of a portion of the airway management system of FIG. 10, illustrating the interface of FIG. 15 and the adjustable seals in an unlocked configuration.

FIG. 17 is an image of the airway management system of FIG. 10 fitted on the head of a patient.

FIG. 18 is a perspective view of another suitable embodiment of first and second rings for the adjustable seal of FIG. 1, illustrating the first and second rings in a locked configuration.

FIG. 19 is a perspective view of the first and second rings of FIG. 18, illustrating the first and second rings in an unlocked configuration.

FIG. 20 is a perspective view of yet another suitable embodiment of first and second rings for the adjustable seal of FIG. 1, illustrating the first and second rings in an unlocked configuration.

FIG. 21 is a side view of the first and second rings of FIG. 20, with a portion of the first and second rings made transparent to illustrate lock features of the first and second rings.

FIG. 22 is a perspective view of still another suitable embodiment of first and second rings for the adjustable seal of FIG. 1, illustrating the first and second seals in a locked configuration.

FIG. 23 is an enlarged view of a portion of the first and second rings indicated by box 23 of FIG. 22, illustrating lock features of the first and second rings.

FIG. 24 is a perspective view of still yet another suitable embodiment of first and second rings for the adjustable seal of FIG. 1, illustrating the first and second rings in an unlocked configuration.

FIG. 25 is a side view of the first and second rings of FIG. 24, illustrating the first and second rings in the unlocked configuration.

FIG. 26 is a perspective view of the first ring of FIG. 22, illustrating engagement grooves in the first ring.

FIG. 27 is a perspective view of the second ring of FIG. 22, illustrating engagement pegs on the second ring.

FIG. 28 is a series of graphs illustrating results of tests that compared air leakage to valve rotations for a plurality of seals.

FIG. 29 is a series of graphs illustrating results of tests that compared water leakage amounts for a plurality of seals.

FIG. 30 is a graph illustrating results of tests that compared air leakage to an inserted device outer diameter for a plurality of seals.

The present embodiments are not limited to the precise arrangements and are instrumentalities shown in the figures. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

The disclosed systems, devices, and methods include an adjustable seal that selectively seals a passage. For example, some embodiments include an adjustable seal that selectively seals around at least one medical instrument associated with an airway management procedure or treatment, including, but not limited to, a bronchoscope, a laryngoscope, or a laryngeal mask airway (LMA). The adjustable seal is adjustable between a number of configurations that allow adjustment of the opening of the passage and allow the passage to be sealed around an object if the object is positioned in the passage or completely close the passage if the passage is not being used. For example, the adjustable seal includes a first ring and a second ring that are rotatable relative to each other to switch the adjustable seal between an opened configuration, a partly opened configuration, and a closed configuration. An elastic flexible member is attached to the first ring and the second ring and selectively extends across the passage as the rings are rotated relative to each other. In some embodiments, the first ring and the second ring include lock features that selectively engage each other to lock the adjustable seal in a desired configuration.

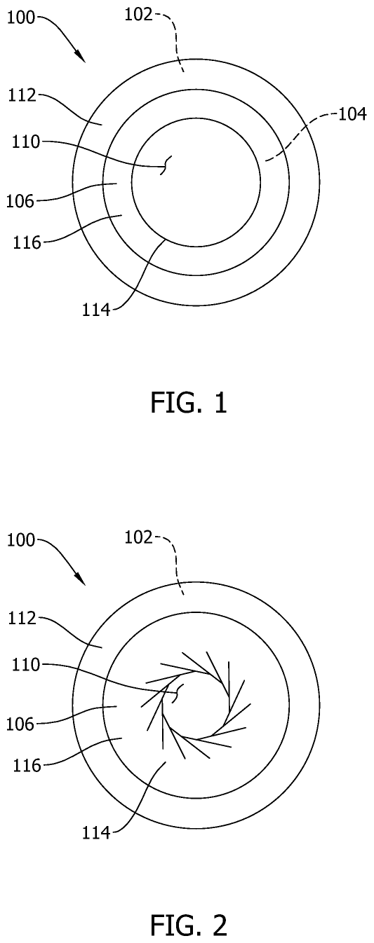

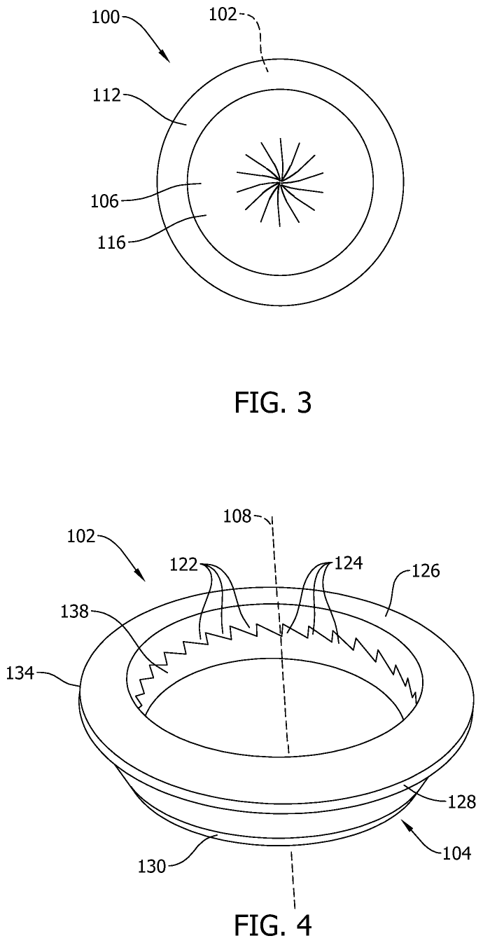

With reference to FIGS. 1-4, an example embodiment of an adjustable seal is generally indicated by 100. The adjustable seal 100 includes a first ring 102, a second ring 104, and a flexible member 106 attached to the first ring 102 and the second ring 104. The first ring 102 and the second ring 104 extend around an axis 108 and define a cavity 110 extending along the axis 108. The adjustable seal 100 is adjustable between different configurations that allow the cavity 110 to be selectively opened/closed and to seal around an object in the cavity 110. For example, the adjustable seal 100 may have a first, open configuration (shown in FIG. 1), a second, partly open configuration (shown in FIG. 2), and a third, closed configuration (shown in FIG. 3). In addition, in the illustrated embodiment, the adjustable seal 100 has a plurality of intermediate configurations in addition to the first, second, and third configurations. As a result, the adjustable seal 100 facilitates selectively closing/opening the cavity 110 to accommodate different objects in the passage and/or to completely close the passage. Moreover, as described further herein, the adjustable seal 100 is simple and easy to switch between the configurations.

The first ring 102 is configured to rotate relative to the second ring 104 to move the adjustable seal between the opened/closed configurations. The flexible member 106 is attached to the first ring 102 and the second ring 104 such that the flexible member 106 selectively opens/closes the cavity 110 as the first ring 102 is rotated relative to the second ring 104. For example, in the illustrated embodiment, the flexible member 106 includes a first end 112 attached to the first ring 102, a second end 114 attached to the second ring 104, and a body 116 extending between the first end 112 and the second end 114. The flexible member 106 is attached to the first ring 102 and the second ring 104 such that the body 116 extends about and along the axis 108 when the adjustable seal 100 is in the opened configuration (FIG. 1). The first end 112 is fixed about the circumference of the first ring 102 such that the first end 112 of the flexible member 106 does not move relative to the first ring 102 as the first ring 102 is rotated relative to the second ring 104. The second end 114 is fixed about the circumference of the second ring 104 such that the second end 114 of the flexible member 106 does not move relative to the second ring 104 as the first ring 102 is rotated relative to the second ring 104. Accordingly, the first end 112 rotates with the first ring 102 relative to the second end 114 as the first ring 102 is rotated relative to the second ring 104. As a result, the flexible member 106 is stretched and twisted between the first end 112 and the second end 114 as the first ring 102 is rotated relative to the second ring 104. If the first end 112 is rotated to a position circumferentially offset from the second end 114, the flexible member 106 extends across and at least partly occludes the cavity 110. To seal the cavity 110, the first end 112 may be rotated to a position in which the flexible member 106 completely closes the passage or to a position in which the flexible member 106 seals against an object in the cavity 110.

The body 116 extends from the first end 112 to the second end 114 and is spaced from the axis 108 when the adjustable seal 100 is in the opened position (FIG. 1). In the exemplary embodiment, the flexible member 106 is a hollow cylinder or sleeve. The flexible member 106 may be attached to the first ring 102 and/or the second ring 104 by positioning the respective end 112, 114 of the flexible member 106 on or around the first ring 102 or the second ring 104 and partially inverting the flexible member 106 such that the flexible member 106 extends over the respective ring 102, 104. In some embodiments, the first end 112, the second end 114, the first ring 102, and/or the second ring 104 may include an adhesive to facilitate attachment of the flexible member 106 to the first ring 102 and/or the second ring 104. In some embodiments, the materials of the flexible member 106 and the rings 102, 104 are selected to provide increased friction between the rings 102, 104 and the flexible member 106 and increase adherence of the flexible member 106 to the rings 102, 104. In alternative embodiments, the flexible member 106 may be secured to the rings 102, 104 using fasteners or any other suitable securement mechanism. In alternative embodiments, the body 116 may include an inner wall and an outer wall that are spaced radially apart such that the inner wall, the outer wall, the first end 112, and the second end 114 collectively define an interior cavity sized to receive the first ring 102 and/or the second ring 104 therein.

Any suitable flexible material may be used to form the flexible member 106. For example, the flexible member 106 may be constructed of an elastic material capable of stretching and twisting and having sufficient resilience to return to an original position after use. The elastic material may have a sufficient strength and/or toughness to resist damage during use. Non-limiting examples of suitable elastic materials include elastic polymers such as latex, silicone, rubber, or nitrile.

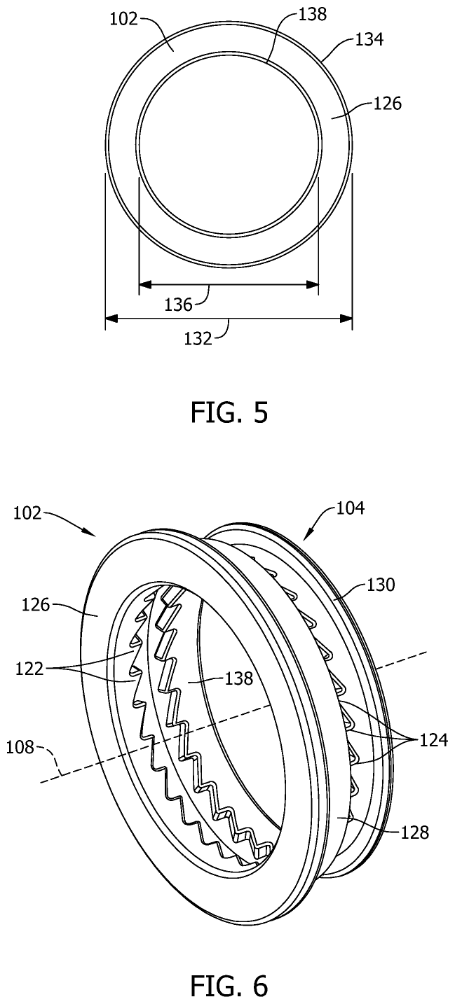

With reference to FIGS. 5-7, the adjustable seal 100 is selectively lockable in one or more configurations (e.g., the opened configuration shown in FIG. 1, the partly open configuration shown in FIG. 2, and the closed position shown in FIG. 3). The first ring 102 includes a plurality of first engagement teeth 122. The second ring 104 includes a plurality of second engagement teeth 124. The first engagement teeth 122 are configured to engage the second engagement teeth 124 to lock the adjustable seal 100 in a desired configuration (i.e., the first ring 102 is not able to rotate relative to the second ring 104 when the first engagement teeth 122 are engaged with the second engagement teeth 124). The adjustable seal 100 is unlocked (i.e., the first ring 102 is able to rotate relative to the second ring 104) when the first engagement teeth 122 are not engaged with the second engagement teeth 124. The first ring 102 may be movable axially (i.e., in a direction parallel to the axis 108) relative to the second ring 104 to engage/disengage the teeth 122, 124 and selectively lock/unlock the adjustable seal 100.

The first engagement teeth 122 and the second engagement teeth 124 extend axially from opposing surfaces of respective rings 102, 104 and are arranged circumferentially about the axis 108. The first engagement teeth 122 and the second engagement teeth 124 are symmetric about the axis 108 and arranged in continuous ring along the entire circumference of the rings 102, 104. The first engagement teeth 122 and the second engagement teeth 124 provide a stepwise adjustment of the adjustable seal 100. In some embodiments, the first ring 102 and the second ring 104 each include at least thirty engagement teeth 122, 124 and the adjustable seal 100 is adjustable in angular increments of 120 or less. The adjustable seal 100 defines a passage with a diameter that is adjustable based on the number and size of the teeth 122, 124. For example, the diameter of the adjustable seal 100 may vary from 1 cm to 50 cm. In further embodiments, the diameter may be greater than 50 cm. In alternative embodiments, the adjustable seal 100 may be adjusted in any suitable manner. For example, in some embodiments, the adjustable seal 100 includes ratchet teeth that allow rotation of the rings 102, 104 in one direction but prevent rotation of the rings 102, 104 in an opposite direction when the adjustable seal 100 is locked. In some embodiments, the adjustable seal 100 has a non-graduated adjustment that provides a nearly infinite number of sealing configurations.

In the exemplary embodiment, the first ring 102 and the second ring are circular and the adjustable seal 100 is generally cylindrical. The first ring 102 and the second ring 104 are concentric when the adjustable seal is in the locked configuration. In alternative embodiments, the first ring 102 and/or the second ring 104 may be rectangular, ovular, triangular, polygonal, or any other suitable shape.

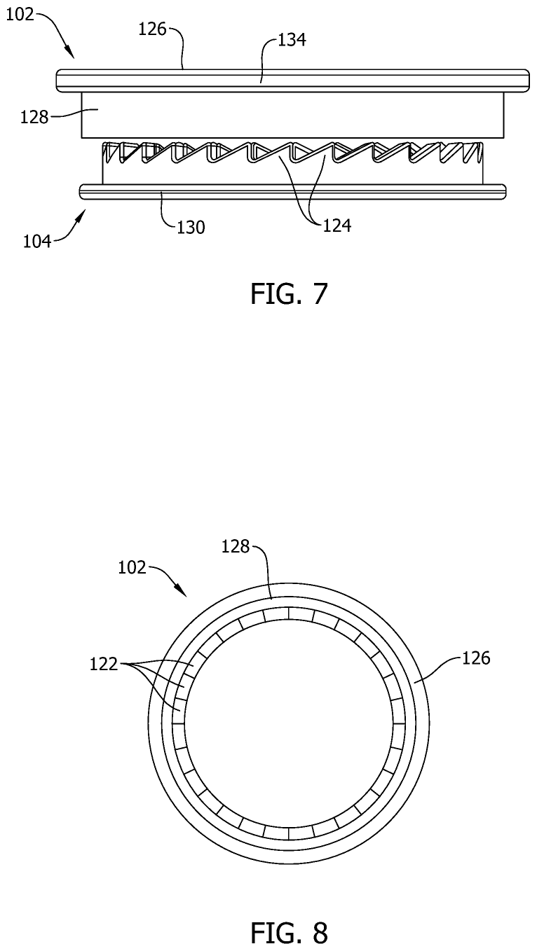

FIG. 8 is a top view of the first ring 102. The first ring 102 includes the engagement teeth 122, an end wall 126, and a circumferential wall 128. The end wall 126 is annular and extends around the axis 108. The engagement teeth 122 and the circumferential wall 128 protrude axially from the end wall 126. The circumferential wall 128 extends around the engagement teeth 122 and axially beyond the engagement teeth 122. Accordingly, the circumferential wall 128 may guide the engagement teeth 124 of the second ring 104 (shown in FIG. 7) into engagement with the engagement teeth 122 of the first ring 102 and inhibit disengagement of engagement teeth 122 and engagement teeth 124.

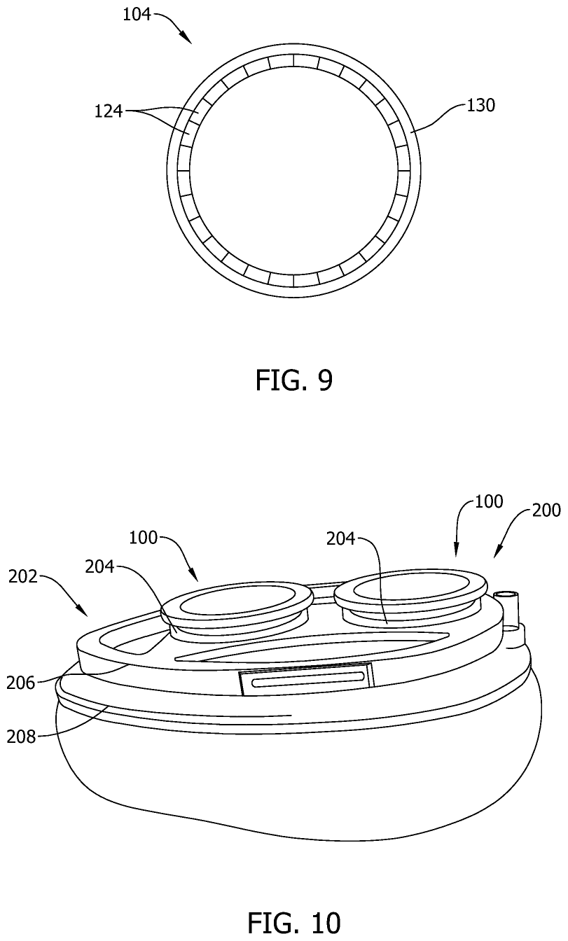

FIG. 9 is a top view of the second ring 104. The second ring 104 includes the engagement teeth 124 and an end wall 130. The end wall 130 is annular and extends around the axis 108. The engagement teeth 124 protrude axially from the end wall 130. The engagement teeth 124 are arranged along an inner edge of the end wall 130 to fit inside the circumferential wall 128 and engage the engagement teeth 122 of the first ring 102.

Referring to FIGS. 8 and 9, the first ring 102 is a single piece and the second ring 104 is a single piece. For example, the engagement teeth 122, an end wall 126, and the circumferential wall 128 of the first ring 102 are integrally formed as a single piece. Also, the engagement teeth 124 and the end wall 130 of the second ring 104 are integrally formed as a single piece. Accordingly, the engagement teeth 122, 124 are formed directly on the walls 128, 130 of the respective rings 102, 104 and the adjustable seal 100 does not require separate locking mechanisms or multiple pieces to be assembled together to form the rings or lock features. As a result, the adjustable seal 100 is simpler to assemble than other seals. In addition, the adjustable seal 100 is simpler to operate and has a decreased risk of failure in comparison to seals with complex locking mechanisms.

The first ring 102 and the second ring 104 may be constructed of any suitable materials. In the exemplary embodiment, the first ring 102 and the second ring 104 are constructed of a rigid plastic that is configured to resist bending as the adjustable seal 100 (shown in FIG. 1) is switched between different configurations. For example, in some embodiments, the first ring 102 and/or the second ring 104 may be constructed of polyethylene terephthalate (PET), polyvinyl chloride (PVC), polyethylene, and/or any similar biomaterial. In some embodiments, the first ring 102 and/or the second ring 104 may be fabricated using 3D printing devices and methods.

Referring to FIGS. 5-9, the adjustable seal 100 has an outer diameter 132 defined by an outer edge 134 of the end wall 126 of the first ring 102 and an inner diameter 136 defined by an inner surface 138 of the first ring 102 and the second ring 104. The inner surface 138 is defined at least in part by the engagement teeth 122, 124. The adjustable seal 100 is sized and shaped to be positioned in a fluid passage to selectively seal the passage. For example, the adjustable seal 100 may be positioned in the passage such that at least the circumferential wall 128 of the first ring 102 engages a body defining the passage and fluid in the passage is able to flow through the cavity 110 along the flexible member 106 and the inner surface 138 of the adjustable seal 100. In addition, the adjustable seal 100 may receive objects extending through the passage in the cavity 110 and the flexible member 106 may selectively seal around the objects. Suitably, the flexible member 106 is configured to seal around objects of different sizes, shapes, and orientation. Accordingly, the adjustable seal 100 is modular and can be used in a number of applications. In addition, the adjustable seal 100 does not require an object to be positioned precisely relative to the axis 108 for the adjustable seal 100 to provide a proper seal around the object.

During operation, the adjustable seal 100 is positioned in a passage to selectively seal the passage. In some embodiments, the adjustable seal 100 is attached to a mask for selectively sealing at least one passage sized to receive an instrument such as an endotracheal tube. For example, an instrument is inserted through the cavity 110 and into the passage. The adjustable seal 100 is switched between the locked configuration and the unlocked configuration and the seal is positioned to at least partly seal the passage. For example, at least one of the first ring 102 and the second ring 104 is moved in an axial direction relative to the other to the switch the adjustable seal 100 between the locked configuration and the unlocked configuration. The engagement teeth 122, 124 disengage from each other when the first ring 102 and the second ring 104 are moved axially apart. When the adjustable seal 100 is in the unlocked configuration, at least one of the first ring 102 and the second ring 104 is rotated relative to the other to cause the flexible member 106 to extend across at least a portion of the passage. In some embodiments, the flexible member 106 extends across the passage and around an instrument extending through the passage to seal the passage. When the adjustable seal 100 is in the desired position (e.g., open, partly open, or closed), the engagement teeth 122, 124 are engaged and the adjustable seal 100 is switched to the locked configuration.

In embodiments, the adjustable seal 100 may include other rings without departing from aspects of the disclosure. For example, in some embodiments, the adjustable seal 100 includes the first and second rings 102, 104, the first and second rings 400, 402, the first and second rings 500, 502, the first and second rings 600, 602, or the first and second rings 700, 702.

FIG. 10 is a perspective view of an airway management system 200 including a mask 202 and a plurality of the adjustable seals 100. The mask 202 defines a plurality of passages 204. The passages 204 are sized to receive an endotracheal tube or other medical instrument and are selectively sealed by the adjustable seals 100 attached to the mask 202. For example, the airway management system 200 may be a laryngeal mask airway (LMA) system, an endotracheal tube (ET) mask system, or any other airway management system. The mask 202 is designed to provide broad coverage of a patient's lower face (i.e. with mouth opened or closed), to provide adequate visualization of the airways through a sheet of clear plastic or other suitable material, and to provide a sealed fit around the patient's face. In patients with difficult airways for intubation or cases in which temporizing measures of ventilation are needed, the mask 202 may be used for quick, easy, and relatively safe airway access and oxygenation. The mask 202 can be fitted over the patient's nose and mouth during tube placement to provide protection throughout the procedure as well as during short-term ventilation.

The mask 202 may include a frame 206 and a cover 208. The frame 206 may be constructed of any suitably stiff biocompatible material including PLA, ABS, and any combination thereof. The cover 208 may be constructed of any suitable biocompatible material with low stiffness and relatively transparent properties including, but not limited to, FEP, PETG, and any combination thereof. The cover 208 and the frame 206 may cooperatively define the passages 204.

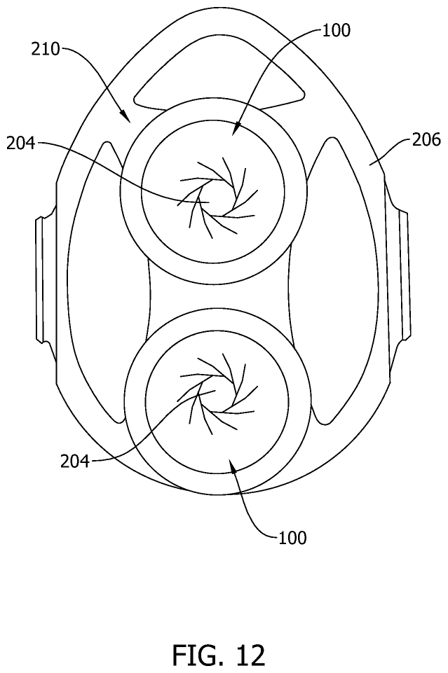

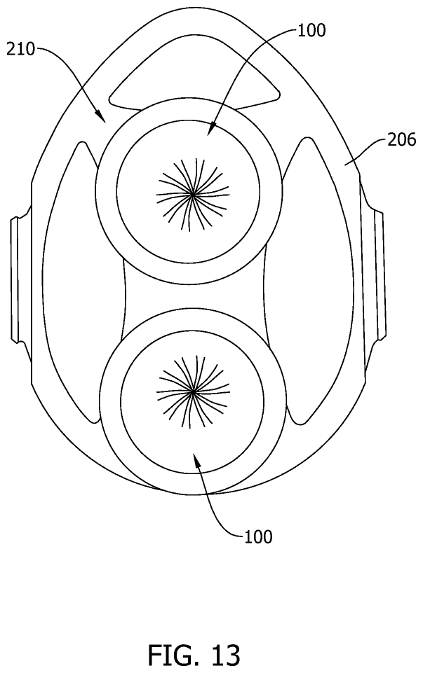

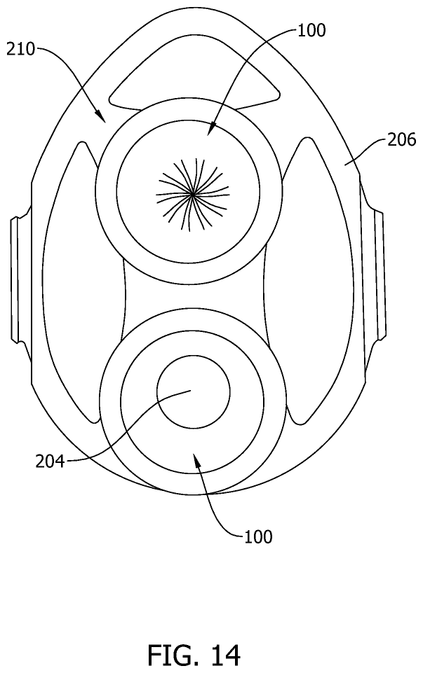

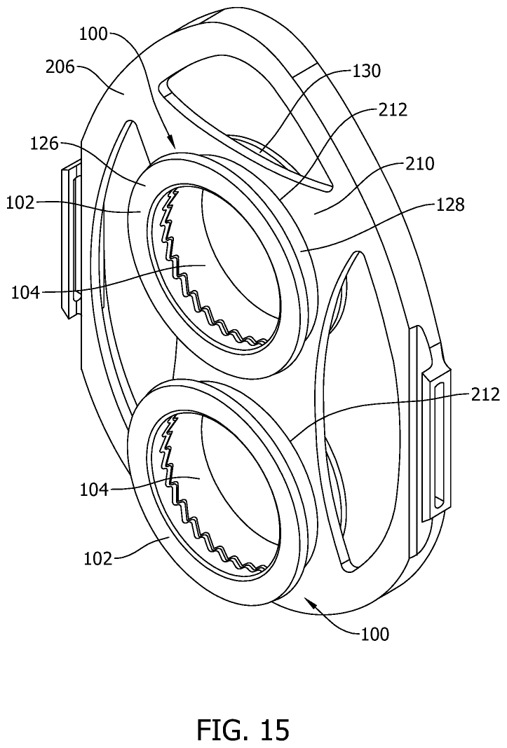

The adjustable seals 100 may be switched between a plurality of configurations to adjust the seal of the passages 204. For example, FIG. 10 illustrates the adjustable seals 100 in an opened configuration. In the opened configuration, the adjustable seals 100 are arranged to allow medical instruments such as air tubes to be positioned in the passages 204. FIG. 12 illustrates the adjustable seals 100 in a partly open configuration. In the partly open configuration, the adjustable seals 100 may seal around the objects positioned in the passages 204. FIG. 13 illustrates the adjustable seals 100 in a closed configuration. FIG. 14 illustrates one of the adjustable seals 100 in an open configuration and another of the adjustable seals 100 in a closed configuration. The adjustable seals 100 may be positioned in different configurations (including configurations not illustrated) to accommodate objects of different sizes, shapes, and orientations. The adjustable seals 100 may allow the objects to be positioned through the passages 204 quickly because the adjustable seals 100 do not require the objects to be positioned in a precise orientation (e.g., the objects do not have to be positioned along a central axis of the passages 204).

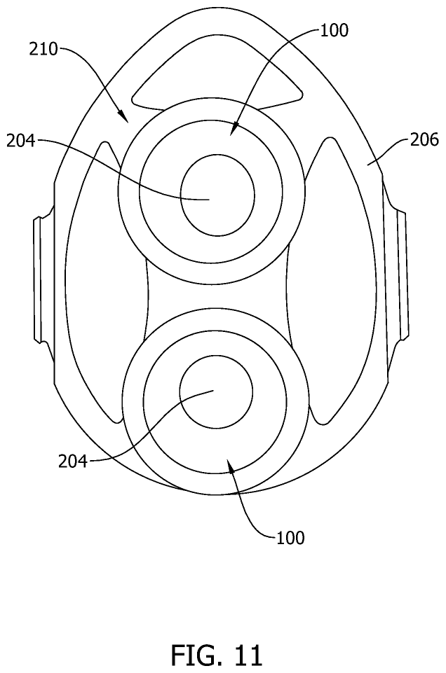

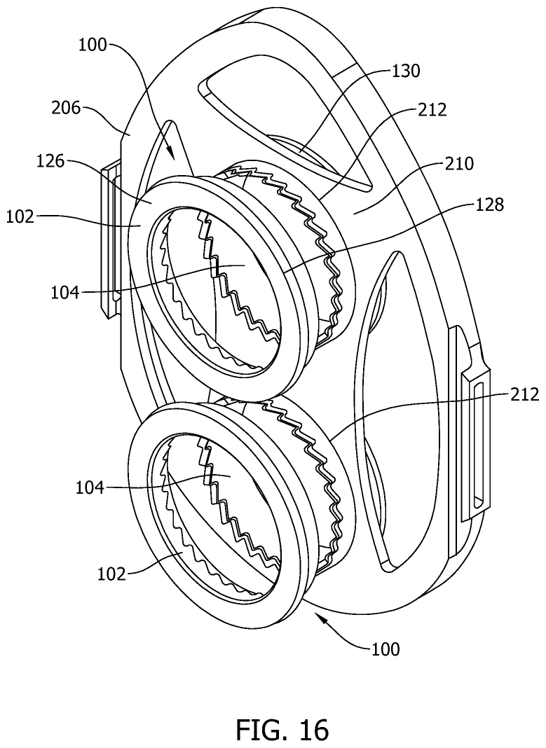

Referring to FIGS. 15 and 16, the frame 206 may include an interface 210 configured to secure the adjustable seals 100 to the mask 202. The interface 210 defines openings 212 and engages the adjustable seals 100 when the adjustable seals 100 are positioned in the openings 212. For example, the interface 210 is configured to extend around the adjustable seals 100 between the end wall 126 of the first ring 102 and the end wall 130 of the second ring 104 and engage the circumferential wall 128 of the first ring 102 when the first ring 102 and the second ring 104 are positioned in the openings 212. The diameter of the openings 212 is less than the diameter of the end wall 126 of the first ring 102 and the diameter of the end wall 130 of the second ring 104. Accordingly, the end walls 126, 130 may inhibit disengagement of the adjustable seal 100 from the interface 210 during use. The adjustable seal 100 may be secured to the interface 210 by inserting the first ring 102 and the second ring 104 into the opening 212 from opposite sides and securing the first ring 102 and the second ring 104 together. The first ring 102 and the second ring 104 may be secured together by attaching the flexible member 106 (shown in FIGS. 11-12) to the first ring 102 and the second ring 104.

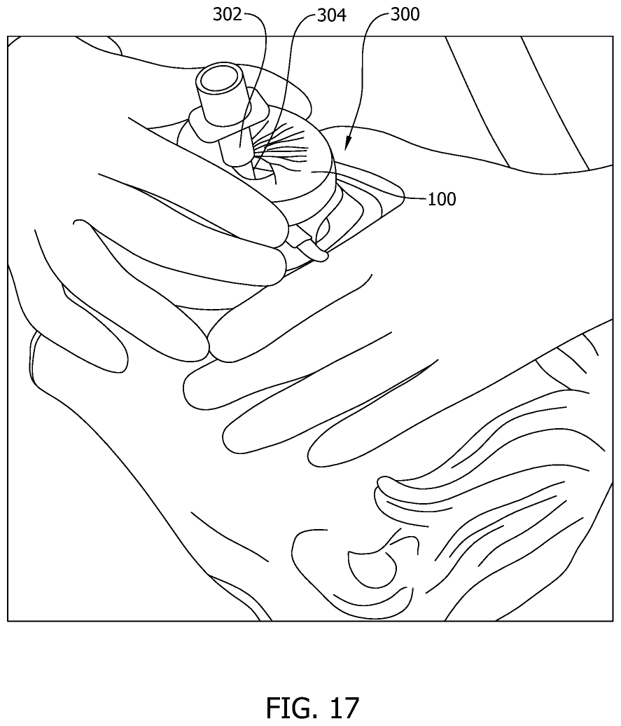

FIG. 17 is an image of a mask 300 fitted onto the head of a patient. The mask 300 may be used to facilitate the placement of an airway for a patient while preventing the release of droplets or aerosolized pathogens from the airway of the patient. The mask 300 is positioned over the nose and mouth of the patient. In some embodiments, the mask 300 may include straps or other features to adjust the fit of the mask 300 on the patient and secure the mask 300 on the patient. The adjustable seal 100 is attached to the mask 300 and allows for an airway device, e.g., an air tube or other device, 302 to be positioned through a passage 304 of the mask 300 to provide an airway for the patient. The adjustable seal 100 provides a seal around the airway device 302 that is positioned in the passage 304 of the mask 300. Accordingly, an air-tight seal may be maintained to prevent the release of droplets and aerosolized pathogens from the patient during placement and use of the mask 300.

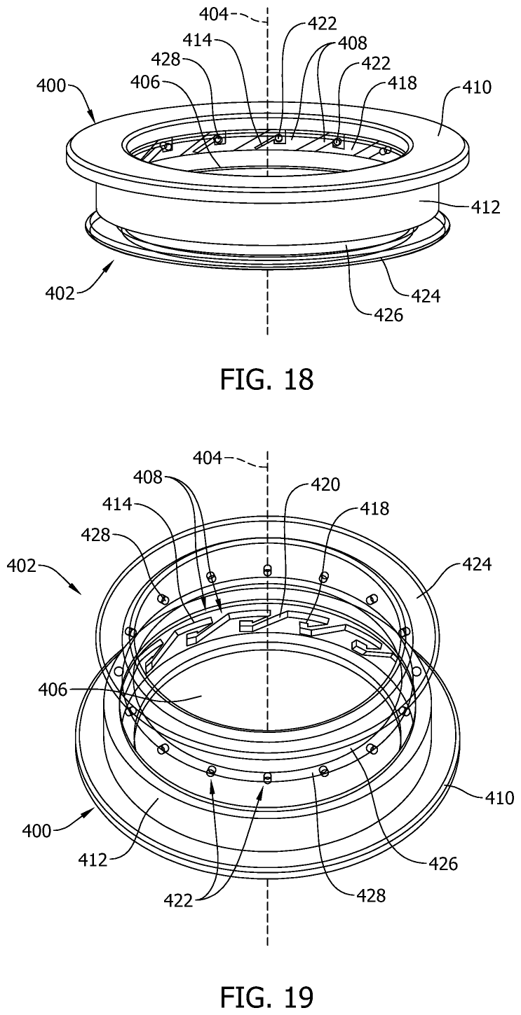

FIG. 18 is a perspective view of an embodiment of a first ring 400 and a second ring 402 for the adjustable seal 100 (shown in FIG. 1), illustrating the first and second rings in a locked configuration. FIG. 19 is a perspective view of the first and second rings of FIG. 18, illustrating the first and second rings in an unlocked configuration. The first ring 400 and the second ring 402 extend around an axis 404 and define a cavity 406 extending along the axis 404. The flexible member 106 shown in FIG. 1 may be attached to the first ring 400 and the second ring 402 and the first ring 400 and the second ring 402 may be rotated relative to each other to switch the adjustable seal 100 between different configurations that allow the cavity 406 to be selectively opened/closed and to seal around an object in the cavity 406.

The first ring 400 includes lock features 408, an end wall 410, and a circumferential wall 412. The end wall 410 is annular and extends around the axis 404. The circumferential wall 412 protrudes axially from the end wall 410. The lock features 408 include protrusions 414 that extend radially inward from the circumferential wall 412 and define grooves 418 that extend circumferentially along the circumferential wall 412. The protrusions 414 includes ramps 420 that are configured to direct lock features on the second ring 402 into or out of the grooves 418 of the first ring 400 when the first ring 400 is moved toward and rotated relative to the second ring 402. The circumferential wall 412 extends around the lock features 408 and axially beyond the lock features. Accordingly, the circumferential wall 412 may guide the lock features 422 of the second ring 402 into engagement with the lock features 408 of the first ring 400 and inhibit disengagement of the lock features 408, 422.

The second ring 402 includes lock features 422, an end wall 424, and a circumferential wall 426. The end wall 424 is annular and extends around the axis 404. The circumferential wall 426 protrudes axially from the end wall 424. The lock features 422 include pegs 428 that protrude radially outward from an outer surface of the circumferential wall 426. The circumferential wall 426 and the pegs 428 are arranged along an inner edge of the end wall 424 to fit inside the circumferential wall 412 of the first ring 400 and for the pegs 428 to engage the lock features 408 of the first ring 400.

The lock features 408 of the first ring 400 are configured to engage the lock features 422 of the second ring 402 to lock the first ring 400 and the second ring 402 in a desired configuration (i.e., the first ring 400 is not able to rotate relative to the second ring 402 when the first lock features 408 are engaged with the second lock features 422). The first ring 400 and the second ring 402 are unlocked (i.e., the first ring 400 is able to rotate relative to the second ring 402) when the first lock features 408 are not engaged with the second lock features 422. The first ring 400 may be rotatable relative to the second ring 402 to engage/disengage the lock features 408, 422 and selectively lock/unlock the first ring 400 and the second ring 402.

The lock features 408, 422 extend radially from opposing surfaces of respective rings 400, 402 and are arranged circumferentially about the axis 404. The protrusions 414 are symmetric about the axis 404 and are arranged in a continuous ring along the entire circumference of the first ring 400. The pegs 428 are symmetric about the axis 404 and are spaced circumferentially about the second ring 402. The protrusions 414 and the pegs 428 provide a stepwise adjustment of the first ring 400 and the second ring 402. For example, the pegs 428 are configured to slide along the ramps and into the grooves 418 to engage the protrusions 414 when the first ring 400 is rotated counter-clockwise relative to the second ring 402. The protrusions 414 are shaped to engage and lock the pegs 428 within the grooves 418. For example, ends of the protrusions 414 form hooks or bayonets that extend at least partly around the pegs 428. The pegs 428 are configured to slide along the ramps and out of the grooves 418 when the first ring 400 is rotated clockwise relative to the second ring 402. In some embodiments, the first ring 400 includes at least ten protrusions and the second ring 402 includes at least ten pegs 428.

In the exemplary embodiment, the first ring 400 and the second ring 402 are circular. The first ring 400 and the second ring 402 are concentric when the first ring and the second ring are in the locked configuration. In alternative embodiments, the first ring 400 and/or the second ring 402 may be rectangular, ovular, triangular, polygonal, or any other suitable shape.

The first ring 400 is a single piece and the second ring 402 is a single piece. For example, the lock features 408, the end wall 410, and the circumferential wall 412 of the first ring 400 are integrally formed as a single piece. In addition, the lock features 422, the end wall 424, and the circumferential wall 426 of the second ring 402 are integrally formed as a single piece.

Referring to FIGS. 18 and 19, the first ring 400 and the second ring 402 may be constructed of any suitable materials. In the exemplary embodiment, the first ring 400 and the second ring 402 are constructed of a rigid plastic that is configured to resist bending as the adjustable seal 100 (shown in FIG. 1) is switched between different configurations. For example, in some embodiments, the first ring 400 and/or the second ring 402 may be constructed of polyethylene terephthalate (PET), polyvinyl chloride (PVC), polyethylene, and/or any similar biomaterial. In some embodiments, the first ring 400 and/or the second ring 402 may be fabricated using 3D printing devices and methods.

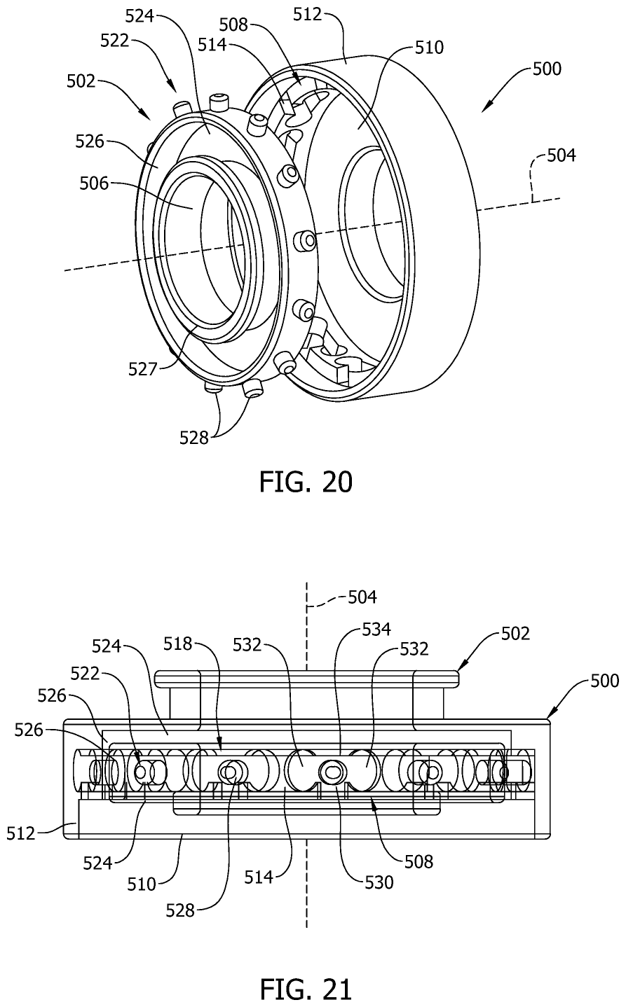

FIG. 20 is a perspective view an embodiment of a first ring 500 and a second ring 502 for the adjustable seal 100 shown in FIG. 1. The first ring 500 and the second ring 502 extend around an axis 504 and define a cavity 506 extending along the axis 504. The flexible member 106 shown in FIG. 1 may be attached to the first ring 500 and the second ring 502 and the first ring 500 and the second ring 502 may be rotated relative to each other to switch the adjustable seal 100 between different configurations that allow the cavity 506 to be selectively opened/closed and to seal around an object in the cavity 506.

The first ring 500 includes lock features 508, an end wall 510, and a circumferential wall 512. The end wall 510 is annular and extends around the axis 504. The circumferential wall 512 protrudes axially from the end wall 510. The lock features 508 include protrusions 514 that extend radially inward from the circumferential wall 512 and define grooves 518. The circumferential wall 512 extends around the lock features 508 and axially beyond the lock features. Accordingly, the circumferential wall 512 may guide the lock features 522 of the second ring 502 into engagement with the lock features 508 of the first ring 500 and inhibit disengagement of the lock features 508, 522.

The second ring 502 includes lock features 522, an end wall 524, an outer circumferential wall 526, and an inner circumferential wall 527. The end wall 524 is annular and extends around the axis 504. The outer circumferential wall 526 protrudes axially from a radially outer edge of the end wall 524 and the inner circumferential wall 527 protrudes axially from a radially inner edge of the end wall 524. The lock features 522 include pegs 528 that protrude radially outward from an outer surface of the outer circumferential wall 526. The outer circumferential wall 526 and the pegs 528 are arranged along the outer edge of the end wall 524 to fit inside the circumferential wall 512 of the first ring 500 and for the pegs 528 to engage the lock features 508 of the first ring 500.

FIG. 21 is a side view of the first and second rings 500, 502, with a portion of the first and second rings 500, 502 made transparent to illustrate the lock features 508, 522 of the first and second rings 500, 502. The lock features 508 of the first ring 500 are configured to engage the lock features 522 of the second ring 502 to lock the first ring 500 and the second ring 502 in a desired configuration (i.e., the first ring 500 is not able to rotate relative to the second ring 502 when the lock features 508 of the first ring 500 are engaged with the lock features 522 of the second ring 502). The first ring 500 and the second ring 502 are unlocked (i.e., the first ring 500 is able to rotate relative to the second ring 502) when the lock features 508 of the first ring 500 are not engaged with the lock features 522 of the second ring 502. The first ring 500 may be rotatable and movable axially relative to the second ring 502 to engage/disengage the lock features 408, 422 and selectively lock/unlock the first ring 500 and the second ring 502.

Referring to FIGS. 20 and 21, the protrusions 514 and the pegs 528 extend radially from opposing surfaces of respective rings 500, 502 and are arranged circumferentially about the axis 504. The protrusions 514 are symmetric about the axis 504 and are arranged in a continuous ring along the entire circumference of the first ring 500. The pegs 528 are symmetric about the axis 504 and are spaced circumferentially about the second ring 502. The protrusions 514 and the pegs 528 provide a stepwise adjustment of the first ring 500 and the second ring 502. For example, the pegs 528 are configured to move into the grooves 518 to engage the protrusions 514 when the first ring 500 is moved axially toward the second ring 502 and rotated clockwise or counter-clockwise relative to the second ring 502. The protrusions 514 are shaped to engage and lock the pegs 528 within the grooves 518. For example, ends of the protrusions 514 form hooks or bayonets that extend at least partly around the pegs 528. In addition, each groove 518 includes a vertical slot 530, a pair of opposed cavities 532 on either side of the vertical slot 530, and a horizontal slot 534 extending between and connecting the cavities 532. Accordingly, the peg 528 is able to be inserted into the vertical slot 530, moved into the horizontal slot 534, and moved in either direction into one of the cavities 532 to capture the peg 528. The pegs 528 are configured to move out of the grooves 518 when the first ring 500 is rotated clockwise or counter-clockwise and moved axially relative to the second ring 502. In some embodiments, the first ring 500 includes at least ten protrusions and the second ring 502 includes at least ten pegs 528.

In the exemplary embodiment, the first ring 500 and the second ring 502 are circular. The first ring 500 and the second ring 502 are concentric when the first ring and the second ring are in the locked configuration. In alternative embodiments, the first ring 500 and/or the second ring 502 may be rectangular, ovular, triangular, polygonal, or any other suitable shape.

The first ring 500 is a single piece and the second ring 502 is a single piece. For example, the lock features 508, the end wall 510, and the circumferential wall 512 of the first ring 500 are integrally formed as a single piece. In addition, the lock features 522, the end wall 524, and the circumferential wall 526 of the second ring 502 are integrally formed as a single piece.

The first ring 500 and the second ring 502 may be constructed of any suitable materials. In the exemplary embodiment, the first ring 500 and the second ring 502 are constructed of a rigid plastic that is configured to resist bending as the adjustable seal 100 (shown in FIG. 1) is switched between different configurations. For example, in some embodiments, the first ring 500 and/or the second ring 502 may be constructed of polyethylene terephthalate (PET), polyvinyl chloride (PVC), polyethylene, and/or any similar biomaterial. In some embodiments, the first ring 500 and/or the second ring 502 may be fabricated using 3D printing devices and methods.

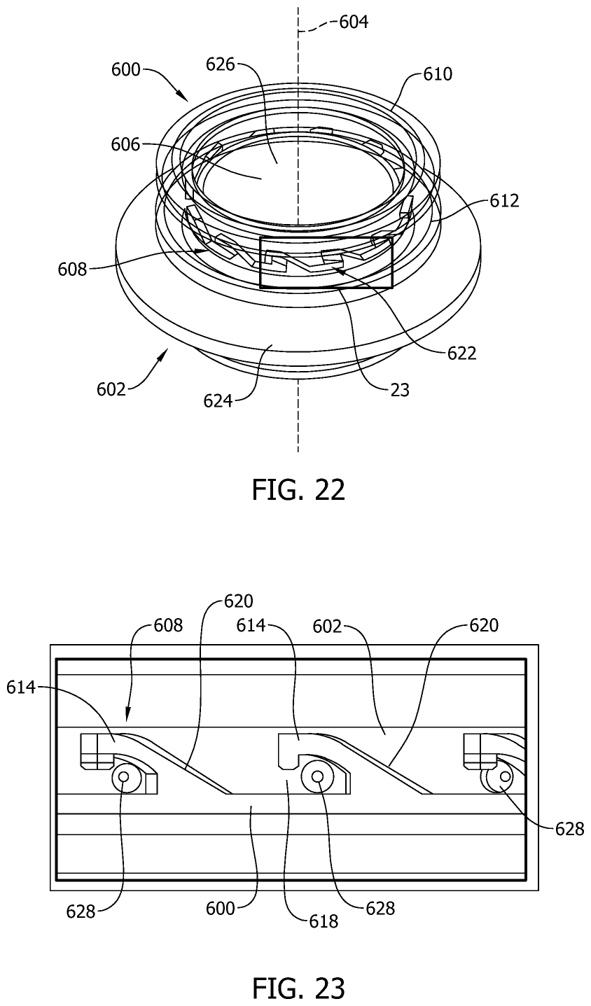

FIG. 22 is a perspective view of an embodiment of a first ring 600 and a second ring 602 for the adjustable seal 100 shown in FIG. 1. The first ring 600 and the second ring 602 extend around an axis 604 and define a cavity 606 extending along the axis 604. The flexible member 106 shown in FIG. 1 may be attached to the first ring 600 and the second ring 602 and the first ring 600 and the second ring 602 may be rotated relative to each other to switch the adjustable seal 100 between different configurations that allow the cavity 606 to be selectively opened/closed and to seal around an object in the cavity 606.

The first ring 600 includes lock features 608, an end wall 610, and a circumferential wall 612. The end wall 610 is annular and extends around the axis 604. The circumferential wall 612 protrudes axially from the end wall 610. The lock features 608 include protrusions 614 that extend radially inward from the circumferential wall 612 and define grooves 618. The protrusions 614 includes ramps 620 that are configured to direct lock features on the second ring 602 into or out of the grooves 618 of the first ring 600 when the first ring 600 is rotated relative to the second ring 602. The circumferential wall 612 extends around the lock features 608 and axially beyond the lock features 608. Accordingly, the circumferential wall 612 may guide the lock features 622 of the second ring 602 into engagement with the lock features 608 of the first ring 600 and inhibit disengagement of the lock features 608, 622.

The second ring 602 includes lock features 622, an end wall 624, and a circumferential wall 626. The end wall 624 is annular and extends around the axis 604. The circumferential wall 626 protrudes axially from the end wall 624. The lock features 622 include pegs 628 that protrude radially outward from an outer surface of the circumferential wall 626. The circumferential wall 626 and the pegs 628 are arranged along an inner edge of the end wall 624 to fit inside the circumferential wall 612 of the first ring 600 and for the pegs 628 to engage the lock features 608 of the first ring 600.

FIG. 23 is an enlarged view of a portion of the first and second rings 600, 602 indicated by box 23 of FIG. 22. The lock features 608 of the first ring 600 are configured to engage the lock features 622 of the second ring 602 to lock the first ring 600 and the second ring 602 in a desired configuration (i.e., the first ring 600 is not able to rotate relative to the second ring 602 when the lock features 608 of the first ring 600 are engaged with the lock features 622 of the second ring 602). The first ring 600 and the second ring 602 are unlocked (i.e., the first ring 600 is able to rotate relative to the second ring 602) when the lock features 608 of the first ring 600 are not engaged with the lock features 622 of the second ring 602. The first ring 600 may be rotatable relative to the second ring 602 to engage/disengage the lock features 608, 622 and selectively lock/unlock the first ring 600 and the second ring 602.

Referring to FIGS. 22 and 23, the protrusions 614 and the pegs 628 extend radially from opposing surfaces of respective rings 600, 602 and are arranged circumferentially about the axis 604. The protrusions 614 are symmetric about the axis 604 and are arranged in a continuous ring along the entire circumference of the first ring 600. The pegs 628 are symmetric about the axis 604 and are spaced circumferentially about the second ring 602. The protrusions 614 and the pegs 628 provide a stepwise adjustment of the first ring 600 and the second ring 602. For example, the pegs 628 are configured to slide along the ramps 620 move and into the grooves 618 to engage the protrusions 614 when the first ring 600 is rotated counter-clockwise relative to the second ring 602. The protrusions 614 are shaped to engage and lock the pegs 628 within the grooves 618. For example, ends of the protrusions 614 form hooks or bayonets that extend at least partly around the pegs 628. The pegs 628 are configured to move out of the grooves 618 when the first ring 600 is rotated clockwise relative to the second ring 602. In some embodiments, the first ring 600 includes at least ten protrusions 614 and the second ring 602 includes at least ten pegs 628.

In the exemplary embodiment, the first ring 600 and the second ring 602 are circular. The first ring 600 and the second ring 602 are concentric when the first ring and the second ring are in the locked configuration. In alternative embodiments, the first ring 600 and/or the second ring 602 may be rectangular, ovular, triangular, polygonal, or any other suitable shape.

The first ring 600 is a single piece and the second ring 602 is a single piece. For example, the lock features 608, the end wall 610, and the circumferential wall 612 of the first ring 600 are integrally formed as a single piece. In addition, the lock features 622, the end wall 624, and the circumferential wall 626 of the second ring 602 are integrally formed as a single piece.

The first ring 600 and the second ring 602 may be constructed of any suitable materials. In the exemplary embodiment, the first ring 600 and the second ring 602 are constructed of a rigid plastic that is configured to resist bending as the adjustable seal 100 (shown in FIG. 1) is switched between different configurations. For example, in some embodiments, the first ring 600 and/or the second ring 602 may be constructed of polyethylene terephthalate (PET), polyvinyl chloride (PVC), polyethylene, and/or any similar biomaterial. In some embodiments, the first ring 600 and/or the second ring 602 may be fabricated using 3D printing devices and methods.

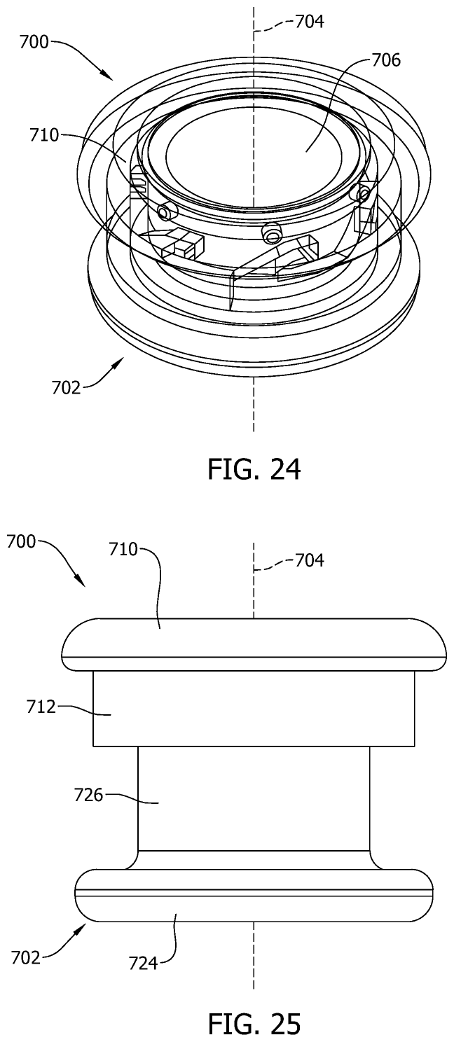

FIG. 24 is a perspective view of an embodiment of a first ring 700 and a second ring 702 for the adjustable seal 100 shown in FIG. 1. The first ring 700 and the second ring 702 extend around an axis 704 and define a cavity 706 extending along the axis 704. The flexible member 106 shown in FIG. 1 may be attached to the first ring 700 and the second ring 702 and the first ring 700 and the second ring 702 may be rotated relative to each other to switch the adjustable seal 100 between different configurations that allow the cavity 706 to be selectively opened/closed and to seal around an object in the cavity 706.

The first ring 700 includes lock features 708, an end wall 710, and a circumferential wall 712. The end wall 710 is annular and extends around the axis 704. The circumferential wall 712 protrudes axially from the end wall 710. The lock features 708 include protrusions 714 that extend radially inward from the circumferential wall 712 and define grooves 718. The protrusions 714 includes ramps 720 that are configured to direct lock features 722 of the second ring 702 into or out of the grooves 718 when the first ring 700 is rotated relative to the second ring 702. The circumferential wall 712 extends around the lock features 708 and axially beyond the lock features. Accordingly, the circumferential wall 712 may guide the lock features 722 of the second ring 702 into engagement with the lock features 708 of the first ring 700 and inhibit disengagement of lock features.

The second ring 702 includes lock features 722, an end wall 724, and a circumferential wall 726. The end wall 724 is annular and extends around the axis 704. The circumferential wall 726 protrudes axially from the end wall 724. The lock features 722 include pegs 728 that protrude radially outward from an outer surface of the circumferential wall 726. The circumferential wall 726 and the pegs 728 are arranged along an inner edge of the end wall 724 to fit inside the circumferential wall 712 of the first ring 700 and for the pegs 728 to engage the lock features 708 of the first ring 700.

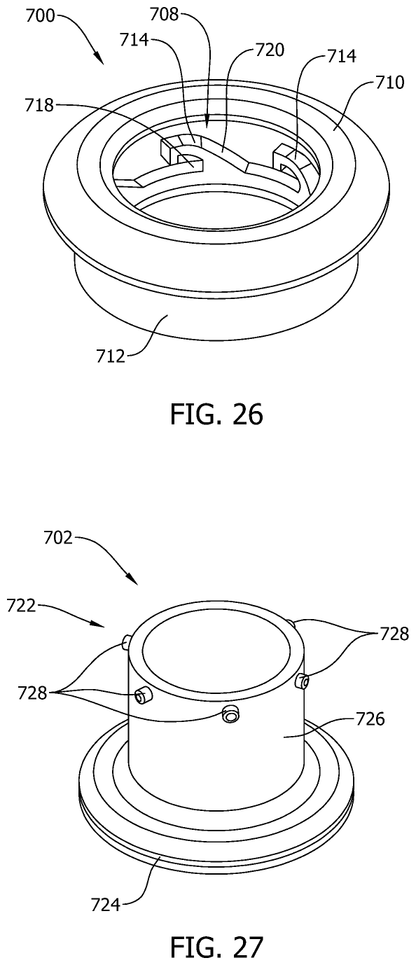

Referring to FIGS. 24-27, the lock features 708 of the first ring 700 are configured to engage the lock features 722 of the second ring 702 to lock the first ring 700 and the second ring 702 in a desired configuration (i.e., the first ring 700 is not able to rotate relative to the second ring 702 when the lock features 708 of the first ring 700 are engaged with the lock features 722 of the second ring 702). The first ring 700 and the second ring 702 are unlocked (i.e., the first ring 700 is able to rotate relative to the second ring 702) when the lock features 708 of the first ring 700 are not engaged with the lock features 722 of the second ring 702. The first ring 700 may be rotatable relative to the second ring 702 to engage/disengage the lock features 708, 722 and selectively lock/unlock the first ring 700 and the second ring 702.

The protrusions 714 and the pegs 728 extend radially from opposing surfaces of respective rings 700, 702 and are arranged circumferentially about the axis 704. The protrusions 714 are symmetric about the axis 704 and are arranged in a continuous ring along the entire circumference of the first ring 700. The pegs 728 are symmetric about the axis 704 and are spaced circumferentially about the second ring 702. The protrusions 714 and the pegs 728 provide a stepwise adjustment of the first ring 700 and the second ring 702. For example, the pegs 728 are configured to slide along the ramps 720 move and into the grooves 718 to engage the protrusions 714 when the first ring 700 is rotated counter-clockwise relative to the second ring 702. The protrusions 714 are shaped to engage and lock the pegs 728 within the grooves 718. For example, ends of the protrusions 714 form hooks or bayonets that extend at least partly around the pegs 728. The pegs 728 are configured to move out of the grooves 718 when the first ring 700 is rotated clockwise relative to the second ring 702. In some embodiments, the first ring 700 includes at least six protrusions 714 and the second ring 702 includes at least six pegs 728.

In the exemplary embodiment, the first ring 700 and the second ring 702 are circular. The first ring 700 and the second ring 702 are concentric when the first ring 700 and the second ring 702 are in the locked configuration. In alternative embodiments, the first ring 700 and/or the second ring 702 may be rectangular, ovular, triangular, polygonal, or any other suitable shape.

The first ring 700 is a single piece and the second ring 702 is a single piece. For example, the lock features 708, the end wall 710, and the circumferential wall 712 of the first ring 700 are integrally formed as a single piece. In addition, the lock features 722, the end wall 724, and the circumferential wall 726 of the second ring 702 are integrally formed as a single piece.

The first ring 700 and the second ring 702 may be constructed of any suitable materials. In the exemplary embodiment, the first ring 700 and the second ring 702 are constructed of a rigid plastic that is configured to resist bending as the adjustable seal 100 (shown in FIG. 1) is switched between different configurations. For example, in some embodiments, the first ring 700 and/or the second ring 702 may be constructed of polyethylene terephthalate (PET), polyvinyl chloride (PVC), polyethylene, and/or any similar biomaterial. In some embodiments, the first ring 700 and/or the second ring 702 may be fabricated using 3D printing devices and methods.

### Test Results

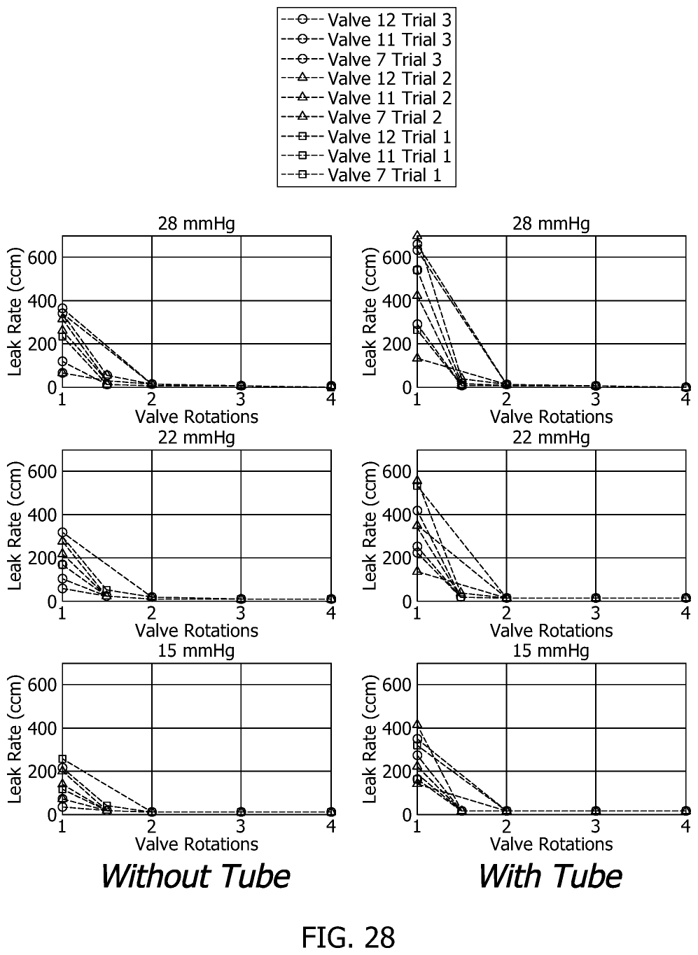

FIG. 28 is a series of graphs illustrating results of tests that compared air leakage to valve rotations for a plurality of seals. For example, embodiments of seals described herein were subjected to various leakage tests. In one example, a valve including the seal 100 shown in FIG. 1 with the first and second rings 700, 702 shown in FIGS. 25-28 was tested. The valve was subjected to pressurized environments (e.g., 28 millimeters (mm) of mercury (Hg), 22 mmHg, and 15 mmHg) and the air leakage through the valve was measured as the valve was rotated. The valve was tested with the seal 100 in a closed position without a tube and with the seal 100 closed about a 13 mm diameter tube inserted through the valve. The seal 100 did not allow any substantial air leakage after the valve was rotated through 1.5 rotations.

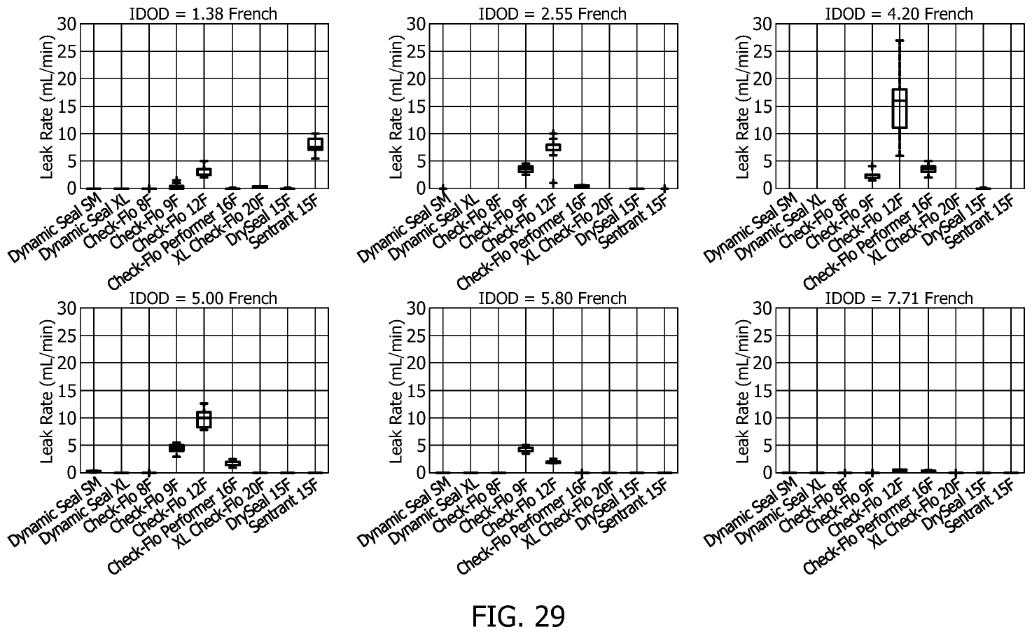

FIG. 29 is a series of graphs illustrating test results that compared water leakage for a plurality of seals. A first tested seal (“Dynamic Seal SM”) was the seal 100 shown in FIG. 1 with the first and second rings 700, 702 shown in FIGS. 25-28. A second tested seal (“Dynamic Seal XL”) was a larger version of the first seal. The first and second seals were compared to other seals.

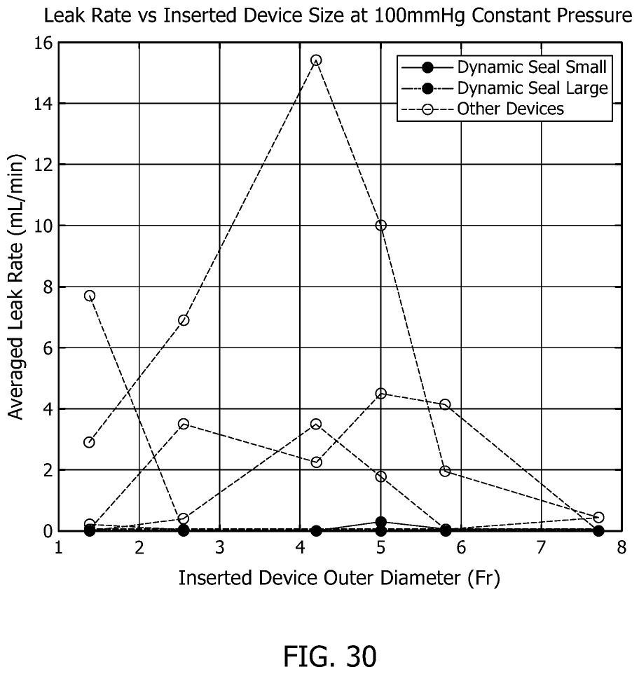

FIG. 30 is a graph comparing the average air leakage to an outer diameter of an inserted device for the first and second tested seals and the other seals. For example, the seals were closed around inserted devices with various diameters (0.46 mm, 0.85 mm, 1.4 mm, 1.67 mm, 1.93 mm, and 2.57 mm) and the leakage rate through the seals were measured in terms of mL/min. The first seal and the second seal prevented substantially all air leakage for all sizes of the inserted devices and performed as well as or better than all prior art seals.

The adjustable seals described herein are well suited for clinical use and provider protection during rapid stabilization in field, during out-of-hospital airway management, and during transportation of persons with known or suspected COVID-19. Additionally, use of the adjustable seals in critically ill patients provides a conduit for quick and easy intubation through a mask without compromising the closed airway system.

The adjustable seals may be used in a number of applications and are not limited to use in the applications described herein. For example, the adjustable seals may be used for apparatus, systems, and methods for hemostatic and hydrostatic valves, hand-assisted laparoscopic surgery, endoscopy procedures, laboratory fume hoods, plumbing systems, heating, ventilation, and air conditioning (HVAC) systems, food packaging, liquid containers, zero-gravity bags, and toys (e.g., fidget toys). The adjustable seals may help modulate the passage of different volumes and rates of fluid flow through fluid passages in a controlled fashion and can reduce pressure or friction on devices or structures passing through the adjustable seals.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An adjustable seal comprising:
a first ring extending about an axis and including a plurality of first lock features, wherein the first ring is a single piece;
a second ring extending about the axis and including a plurality of second lock features, wherein the second ring is a single piece, the first ring and the second ring cooperatively defining a passage extending along the axis, wherein the adjustable seal has a locked configuration in which the second lock features engage the first lock features and prevent movement of the first ring relative to the second ring, and an unlocked configuration in which the first ring is able to rotate relative to the second ring; and
a flexible member attached to the first ring and the second ring, wherein rotation of the first ring relative to the second ring in the unlocked configuration of the adjustable seal causes the flexible member to extend across at least a portion of the passage defined by the first ring and the second ring.

2. The adjustable seal of claim 1, wherein the first lock features include protrusions that define grooves that extend about the axis, and wherein the second lock features include pegs that extend axially from a surface of the second ring, the grooves are sized and shaped to receive the pegs when the adjustable seal is in the locked configuration.

3. The adjustable seal of claim 2, wherein the first ring includes an end wall and a circumferential wall that protrudes axially from the end wall, wherein the protrusions extend radially from and circumferentially along the circumferential wall.

4. The adjustable seal of claim 3, wherein the second ring includes an end wall and a circumferential wall protruding axially from the end wall, wherein the pegs extend radially from the circumferential wall of the second ring.

5. The adjustable seal of claim 1, wherein the first ring includes a circumferential wall that extends axially beyond the first lock features to guide the second lock features of the second ring into engagement with the first lock features of the first ring.

6. The adjustable seal of claim 1, wherein the plurality of first lock features include a plurality of first teeth extending axially from an outer surface of the first ring in a first direction, and wherein the plurality of first lock features include a plurality of second teeth extending axially from an outer surface of the second ring in a second direction opposite the first direction.

7. The adjustable seal of claim 1, wherein the first ring and the second ring are circular.

8. The adjustable seal of claim 1, wherein the first ring and the second ring are constructed of a rigid material.

9. The adjustable seal of claim 1, wherein the flexible member comprises an elastic sleeve that is attached around circumferences of the first ring and the second ring.

10. An airway management system comprising:
a mask sized to cover a nose and mouth of a person, wherein the mask defines at least one passage sized to receive an instrument, the at least one passage extending along an axis;
at least one seal attached to the mask for selectively sealing the at least one passage, the at least one seal comprising:
a plurality of concentric rings and a flexible member attached to the concentric rings, the seal having a locked configuration and an unlocked configuration, each concentric ring including a plurality of lock features that engage each other to lock rotational movement of the concentric rings when the seal is in the locked configuration; and
a flexible member attached to the plurality of concentric rings, wherein rotation of the concentric rings when the seal is in the unlocked configuration causes the flexible member to extend across at least a portion of the passage defined by the mask and seal against the instrument if the instrument is positioned in the passage.

11. The airway management system of claim 10, wherein the at least one passage comprises a first passage and a second passage, and wherein the at least one seal includes a first seal for selectively sealing the first passage and a second seal for selectively sealing the second passage.

12. The airway management system of claim 10 further comprising an endotracheal tube extending through the passage.

13. The airway management system of claim 10, wherein the flexible member comprises an elastic sleeve that is attached around circumferences of the plurality of concentric rings.

14. The airway management system of claim 10, wherein each ring of the plurality of concentric rings includes an end wall and a circumferential wall that protrudes axially from the end wall, wherein the lock features extend radially from the circumferential wall.

15. The airway management system of claim 14, wherein the circumferential wall extends axially beyond the lock features.

16. A method of sealing a passage, the method comprising:
positioning an adjustable seal in a passage, the adjustable seal including a plurality of concentric rings and a flexible member attached to the concentric rings, the adjustable seal having a locked configuration and an unlocked configuration, each concentric ring including a plurality of lock features that engage each other to lock rotational movement of the concentric rings when the adjustable seal is in the locked configuration;
moving at least one of the concentric rings in an axial direction relative to another of the concentric rings to switch the adjustable seal between the locked configuration and the unlocked configuration; and
rotating at least one of the concentric rings when the adjustable seal is in the unlocked configuration to cause the flexible member to extend across at least a portion of the passage.

17. The method of claim 16, further comprising engaging the lock features of the plurality of concentric rings to lock the adjustable seal in the locked configuration after rotating at least one of the concentric rings when the adjustable seal is in the unlocked configuration to cause the flexible member to extend across at least a portion of the passage.

18. The method of claim 17, wherein engaging the lock features of the plurality of concentric rings comprises positioning pegs into grooves that are sized and shaped to receive the pegs when the adjustable seal is in the locked configuration.

19. The method of claim 16, wherein positioning the adjustable seal in the passage comprises attaching the adjustable seal to a mask sized to cover a nose and mouth of a person, wherein the mask defines at least one passage sized to receive an instrument, at least one seal attached to the mask for selectively sealing the at least one passage.

20. The method of claim 19, further comprising inserting an endotracheal tube into the passage, wherein the adjustable seal is configured to seal the at least one passage about the endotracheal tube.
